(19) United States
(12) Patent Application Publication
(10) Pub. No.: US 2006/0177349 A1
(43) Pub. Date: Aug. 10, 2006

(54) Chemical sensor

(76) Inventors: Jacob Thaysen, Kobenhavn N. (DK); Patrick Richard Scheeper, Naerum (DK)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/549,316

(22) PCT Filed: Mar. 18, 2004
(86) PCT No.: PCT/DK04/00185

§ 371 Date: Sep. 16, 2005

(30) Foreign Application Priority Data
Mar. 18, 2003 (DK) PA200300427

(51) Int. Cl. G01N 33/00

(52) U.S. Cl. 422/82.02; 422/98

(57) ABSTRACT

The invention relates to a chemical sensor comprising at least one cantilever sensor unit with a piezoresistive element for direct read out. The sensor comprises a primary substrate carrying the cantilevers and with a primary substrate carrying the cantilevers and with a primary cavity and a primary connecting surface at least partly surrounding said cavity. The cantilevers protrude into the primary cavity. The piezoresistive elements are electrically connected to primary connecting pads on the primary connecting surface. The sensor also comprises a secondary connecting pads, corresponding to the primary connecting pads, on a secondary connecting surface corresponding to the primary connecting surface. The primary connecting surface and the secondary connection surface are mounted to each other so that said primary connecting pads and said secondary connecting pads are direct mounted to each other, preferably in a flip chip mounting.

### FIELD OF THE INVENTION

The present invention relates to a chemical sensor comprising one or more sensor units in the form of cantilevers with a piezoresistive element and means for applying a voltage over said piezoresistive element.

### BACKGROUND OF THE INVENTION

Sensors of the above type are well known from the literature and are e.g. disclosed in WO 00/66266.

From stress formation studies in ambient and aqueous environments, micrometer-sized cantilevers with optical read-out have proven very sensitive as described in the articles Berger, R., Gerber, Ch., Lang, H. P. & Gimzewski, J. K. Micromechanics: A toolbox for femtoscale science: “Towards a laboratory on a tip”. Microelectronic Engineering. 35, 373-379 (1997), and O'Shea, S. J., Welland, M. E. Atomic force Microscopy stress sensors for studies in liquids. J. Vac. Sci. Technol. B. 14, 1383-1385 (1996).

A particularly promising application is for detection of molecular interaction, where capture molecules are immobilized on the surface of the cantilever. A surface stress is induced when target molecules bind to the capture molecules on the cantilever. The surface stress change obtained due to the molecular interaction can be detected by integrating a stress sensitive piezoresistor into the cantilever. By integrating the piezoresistor in a Wheatstone bridge, the resistance change due to the surface stress change is transformed into a change in voltage. Other types of mechanical sensors that are sensitive to surface stress include micro bridges and membranes.

Basically, a biochemical reaction at the cantilever surface can be monitored as a bending or stretch of the cantilever due to a change in the surface stress. Surface stress changes in self-assembled alkanethiols on gold have earlier been measured in air by this technique, and surface stress changes of approximately 10⁻⁵ N/m can be resolved by cantilever-based methods. This sensor principle has a wide range of applications in the detection of specific biomolecules as well as in real time local monitoring of chemical and biological interactions.

Cantilever-based sensors with integrated piezoresistive read-out are described by Thaysen, J., Boisen, A., Hansen, O. & Bouwstra, S. AFM probe with piezoresistive read-out and highly symmetrical Wheatstone bridge arrangement. Proceedings of Transducers '99, 1852-1855 (Sendai 1999). Hereby the stress changes on the cantilever sensors can be registered directly by the piezoresistor. Each sensor has a built-in reference cantilever, which makes it possible to subtract background drift directly in the measurement. The two cantilevers are connected in a Wheatstone bridge, and the stress change on the measurement cantilever is detected as the output voltage from the Wheatstone bridge.

Sensors comprising sensor units in the form of cantilevers with a piezoresistive element for direct read out are relatively small. This is often advantageous when measuring in liquid samples, as the amount of sample necessary to perform a measurement can be relatively small. Thus it is of course also important to be able to handle such small sample i.e. it is important to provide a small liquid chamber where the sample can be contacted with the cantilever or cantilevers.

In the prior art sensors, e.g. as disclosed in “cantilever-based bio-chemical sensor integrated in a microliquid handling system” by J. Thaysen et al. 0-7803-5998-4/01 2001 IEEE, the sensor unit is mounted on a plate so that the cantilevers protrude into a liquid channel and the piezoresistive elements are wire bonded. The resulting chip and the liquid chamber thereby become relatively large compared to the size of the cantilever.

### SUMMARY OF THE INVENTION

The object of the present invention is to provide a chemical sensor which can be produced in a simple way, and where it is possible to optimise the size of the Fluid (liquid or gas, preferably liquid) chamber relative to the size of the cantilever.

An additional objective of the invention is to provide a sensor which may comprise an array of piezoresistor sensor units for direct read-out, wherein the amount of fluid, preferably liquid, necessary to perform the measurements is relatively small and simultaneously the risk of short circuiting is low.

These and other objectives have been achieved by the invention as defined in the claims.

### DISCLOSURE OF THE INVENTION

The term “chemical sensor” includes all sensor where a chemical action is involved e.g. a chemical attraction, or bonding both specific and non-specific. The chemical sensor may be used in the detection of any kinds of chemicals, such as inorganic and organic components including but not limited to inorganic compositions, explosives, components of biological origin, such as human origin or synthetic components resembling these biomolecules, tissues, cells, body fluids, blood components, microorganism, and derivatives thereof, or parts thereof, such as one or more biomolecules of microbial, plant, animal or human origin or synthetic molecules resembling them, more specifically biomolecules such as proteins, nucleic acids, such as RNA, DNA, cDNA, LNA, PNA, oligonucleotides, peptides, hormones, antigen, antibodies, lipids and complexes including one or more of these molecules, said biomolecule preferably being selected from the group consisting of proteins and protein complexes.

The sensor may be used for detection a chemical component in a fluid, such as a gas or a liquid. Preferably the sensor is a liquid sensor for detection a chemical component in a liquid.

The chemical sensor of the invention comprises one or more sensor units, such as 1, 2, 5, 10, 50 or even more e.g. up to 100 or 300 sensor units, such as it is disclosed in WO 0066266, PCT/DK/0200779, PCT/DK/0300117, PCT/DK/0300042, PCT/DK/0300086, DK PA 2002 00884, DK PA 2002 01221, and DK PA 2002 00068, which with respect to the disclosure concerning sensor unit structure in the form of cantilevers (also including cantilever like structures and polycantilevers as disclosed in PCT/DK/0300086, PCT/DK/0300042 and DK PA 2002 00068), size of the cantilever, materials, and type of capture surface are hereby incorporated by reference.

The one or more sensor units are in the form of cantilevers. This should be interpreted so that the sensor units may have any cantilever like shape e.g. as the cantilevers described in PCT/DK/0300042 and DK PA 2002 00068. The term “cantilever” is defined as a sheet formed unit linked to a substrate along one or two opposite edge lines. The term “cantilever” thus also includes a bridge as well as a traditional rectangular or leaf-shaped cantilever.

In one embodiment, the sensor unit shaped as a cantilever with a longitudinal direction is linked in both of its longitudinal endings to form a cantilevered bridge.

In another embodiment, the cantilever is a traditional rectangular or leaf-shaped cantilever linked to and protruding from one substrate. This shape is further disclosed in DK PA 2002 00068

The thickness of the cantilever may preferably be between 0.1 and 25 μm, more preferably between 0.3 and 5 μm, such as about 1 μm. The length and width may e.g. be up to about 500 μm, more preferably up to about 100 μm, such as about 50 μm, and the width may e.g. between 0.05 and 0.5 times its length.

Each of the cantilevers comprises a piezoresistive element for direct read out e.g. as disclosed in WO 0066266, PCT/DK/0200779, PCT/DK/0300117, PCT/DK/0300042, PCT/DK/0300086, DK PA 2002 00884, DK PA 2002 01221, and DK PA 2002 00068.

The piezoresistor may be any kind of resistor capable of changing resistivity due to a deformation provided by deflecting and/or stretching of the sensor unit. Piezoresistors are well known in the art and are e.g. described in the following publications which are hereby incorporated by reference: U.S. Pat. No. 6,237,399, U.S. Pat. No. 5,907,095, Berger, R. et al. Surface stress in the self-assembly of alkanethiols on gold. Science. 276, 2021-2024 (1997); Berger, R., Gerber, Ch., Lang, H. P. & Gimzewski, J. K. Micromechanics: A toolbox for femtoscale science: “Towards a laboratory on a tip”. Microelectronic Engineering. 35, 373-379 (1997); Thaysen, J., Boisen, A., Hansen, O. & Bouwstra, S. AFM probe with piezoresistive read-out and highly symmetrical Wheatstone bridge arrangement. Proceedings of Transducers '99,1852-1855 (Sendai 1999); Boisen A., Thaysen J., Jensenius H., & Hansen, O. Environmental sensors based on micromachined cantilevers with integrated read-out. Ultramicroscopy, 82, 11-16 (2000).

Preferred piezoresistors include piezoresistors of a material selected from the group consisting of one or more of the materials silicon (including polysilicon and single crystal silicon), metal or metal containing composition, e.g. gold, AlN, Ag, Cu, Pt and Al conducting polymers, such as doped octafunctional epoxidized novalac e.g. doped SU-8, and composite materials with an electrically non-conducting matrix and a conducting filler, wherein the filler preferably is selected from the group consisting of polysilicon, single crystal silicon, metal or metal containing composition, e.g. gold, AlN, Ag, Cu, Pt and Al, semi-conductors, carbon black, carbon fibres, particulate carbon, carbon nanowires, silicon nanowires.

Even though the sensor in the following mainly is described with one cantilever it should be understood that the sensor could comprise several as mentioned above, e.g. arranged in a row or several rows.

The sensor comprises a primary and a secondary substrate. The primary substrate comprises a primary cavity and the sensor unit or units are connected to and protruding from said primary substrate and into said primary cavity. The primary cavity thus constitutes the whole or part of a chamber or channel for a fluid sample, where the fluid sample can come into contact with the cantilever(s). This chamber/channel is also denoted an interaction chamber.

The primary substrate may in principle be of any material and shape as disclosed in WO 0066266, PCT/DK/0200779, PCT/DK/0300117, PCT/DK/0300042, PCT/DK/0300086, DK PA 2002 00884, DK PA 2002 01221, and DK PA 2002 00068.

In one embodiment, the primary substrate comprises one or more of the materials selected from the group consisting of silicon (including polysilicon and single crystal silicon), silicon nitride, silicon oxide, metal, metal oxide, glass and polymer, wherein the group of polymers preferably includes epoxy resin e.g. an octafunctional epoxidized novalac, polystyrene, polyethylene, polyvinyl acetate, polyvinylchloride, polyvinylpyrrolidone, polyacrylonitrile, polymethylmetacrylate, polytetrafluoroethylene, polycarbonate, poly-4-methylpentylene, polyester, polypropylene, cellulose, nitrocellulose, starch, polysaccharides, natural rubber, butyl rubber, styrene butadiene rubber and silicon rubber.

In one embodiment, the primary substrate is based on silicon, said primary cavity being in the form of an etched cavity forming a recess under the one or more cantilever(s), e.g. as disclosed in WO 0066266.

In order to have good processability, it may be desired that the primary substrate is of or comprises a material which can act as a photo resist. Preferred materials include an epoxy resin, preferably selected from the group consisting of epoxy functional resin having at least two epoxy groups, preferably an octafunctional epoxidized novalac. Particularly preferred materials are described in U.S. Pat. No. 4,882,245, which are hereby incorporated by reference. The most preferred material is the octafunctional epoxidized novalac which is commercially available from Celanese Resins, Shell Chemical, MicroChem Inc. under the trade name SU-8, and from Softec Microsystems under the trade name SM10#0.

Basically, it is preferred that the cantilevers are based on a material included in the primary substrate or preferably based on the same material as that of the primary substrate. If the sensor unit and the primary substrate are made in one piece, it is naturally based on the same material, but the sensor unit and the primary substrate may include one or more layers of material not included in the other part. To be based on a material means in the invention that at least 75%, preferably at least 90% by volume is constituted by this material.

A primary connecting surface at least partly surrounds the primary cavity. In one embodiment, the primary connecting surface totally surrounds the primary cavity.

The primary connection cavity may in one embodiment be close to the border of the cavity, such as at a distance of 20 μm or less, such as 10 μm or less, such as 5 μm or less. In one embodiment, the primary connection surface is at a distance from the primary cavity border of between 25 and 500 μm, such as between 50 and 150 μm. In one embodiment, the primary connection surface has one or more areas which are close to the primary cavity border e.g. at a distance of 20 μm or less, such as 10 μm or less, such as 5 μm or less, and one or more areas where the primary connection surface is at a larger distance from the primary cavity border e.g. a distance of between 25 and 500 μm, such as between 50 and 150 μm. These areas of the primary connection surface may be in direct contact with each other or one or more of them may be separated from the other areas.

Thus, thanks to the invention it is possible in a very simple and inexpensive manner to produce very small sensors of the above mentioned type, without the risk of blocking or obstructing the cavity.

In one embodiment, the primary connection surface is extending around the primary cavity in an unbroken ring. In this embodiment, the primary connection may further comprise additional areas separate from the ring formed primary connection area for mechanical stabilization of the sensor.

In one embodiment, the primary connecting surface is in the form of two primary connecting surface sections separated from each other and extending along the border of the primary cavity.

In one embodiment, the primary connecting surface is in the form of two primary connecting surface sections separated from each other and extending along opposite border of the primary cavity in the form of a channel.

The primary connecting surface comprises connection pads, such as two or more, e.g. two connection pads for each piezoresistive element. The piezoresistive element or elements is/are electrically connected to these primary connecting pads on the primary connecting surface. This may e.g. be performed using techniques as disclosed in WO 0066266, PCT/DK/0300117 or PCT/DK/0300042.

The primary connection surface may further comprise a barrier line which extends partly or totally around the primary cavity and has the purpose of stopping or blocking a filler, such as a glue, from overflowing the cavity barrier and filling up the cavity. The barrier line is in the form of a barrier wall, a barrier ditch or both a barrier wall and a barrier ditch.

In one embodiment comprising a barrier line, the barrier line is in the form of a barrier ditch. The barrier ditch may e.g. have a depth of up to about 100 μm, such as up to about 50 μm, such as up to about 25 μm. In one embodiment the barrier have a width of up to about 100 μm, such as between 5 and 50 μm, such as between 10 and 25 μm. The depth and the width may vary along the length of the line.

In one embodiment the barrier line is in the form of a barrier wall. This barrier wall may in principle be of any type of material which is not soluble in or react with the liquid sample to be tested. In one embodiment, the barrier wall is of glass or a metal e.g. a soldering material. In another embodiment, the barrier wall is of a polymer e.g. a glue, which essentially is not soluble in the solvent of the underfiller, that means that the barrier wall of polymer is not dissolved when the underfiller is applied. The barrier wall may preferably have a height of 50 μm or less, such as 20 μm or less, such as 10 μm or less, such as 10 μm or less.

The barrier line further more has the effect of providing an improved insulation of the primary and secondary connection surfaces. Using the invention such barrier line is very simple to establish while simultaneously avoiding blocking and/or obstructing the primary cavity.

In embodiments, where the mounting is performed with bumps the barrier wall preferably has a height which is less than the height of the bumps, preferably about 0.5 times the height of the bumps or less.

In one embodiment, the barrier wall may be placed relatively close to the primary cavity, such as at a distance of 20 μm or less, such as 10 μm or less, such as 5 μm or less. In another embodiment the barrier wall may be placed larger distance to the primary cavity, such as at a distance of 20 μm or more, such as 50 μm or more, such as 75 μm or more, or the distance may vary along the length of the barrier wall.

The secondary substrate comprises secondary connecting pads corresponding to the primary connecting pads, on a secondary connecting surface corresponding to the primary connecting surface. The primary connecting surface and said secondary connection surface are mounted to each other so that the primary connecting pads and the secondary connecting pads are directly mounted to each other, preferably in a flip chip mounting.

In one embodiment, wherein either the connection pads on the primary pads or the secondary pads comprise a bump, the connecting pads are connected to each other using soldering.

In one embodiment, wherein either the connection pads on the primary pads or the secondary pads comprise a bump, the connecting pads are connected to each other using an electrically conducting glue e.g. a thermoplastic glue.

The primary and the secondary connecting surfaces, respectively, are the surfaces of the primary substrate that are in tight connection with the secondary substrate and the surface of the secondary substrate that is in tight connection with the primary substrate. The connection is in one embodiment constituted by the connection pads and adhesive.

By the terms “directly mounted to each other” is meant that the connection pads, e.g. in the form of bumps of a conducting material, are directly electrically connected to each other without intermediate wiring. The bump may e.g. be performed by soldering material and the connection may in one embodiment be performed by soldering the pads together.

In one embodiment, the secondary connection surface may further comprise a barrier line which extends partly or totally around a line corresponding to the border of the primary cavity. This barrier line may be as described above. In one embodiment, the barrier line should preferably be placed relatively closely around a line corresponding to the border of the primary cavity, such as at a distance of 20 μm or less, such as 10 μm or less, such as 5 μm or less. In one embodiment, the barrier line extends totally or partly around a cavity or an opening in the secondary substrate.

The secondary substrate may preferably comprise electrical communication lines capable of providing an electrical connection between a power supply and the piezoresistive element(s), to thereby apply a voltage over the piezoresistive element(s).

In one embodiment, the primary connecting pads and the secondary connection are directly mounted to each other in a flip chip mounting. Flip chip mountings are generally known in the art for electrically mounting a die and a package carrier to each other e.g. for mounting a chip to a printed circuit board.

In one embodiment, the secondary substrate is a printed circuit board. The printed circuit board may preferably be a micro circuit board having the dimension of about 50×50 mm or smaller, such as about 15×30 mm or smaller, such as about 6×6 mm or smaller. The printed circuit area mounted to the primary substrate may in one embodiment constitute 100 mm² or less, such as around 50 mm² or less, such as around 36 mm² or less.

In one embodiment the secondary substrate is a micro chip e.g. with maximal dimensions of about 5000 μm or less, such as about 1000 μm or less, such as about 1000 μm or less. In one embodiment, the secondary substrate is a micro chip which comprises electrical connection lines for connection to a power supply.

In one embodiment, the secondary substrate may e.g. be a combined circuit board and flow chip. The circuit board may e.g. comprise an integrated or mounted flow chip for bringing and optionally withdrawing a fluid sample from the sensor.

The term flow chip is herein used to designate a fluid flow chip, i.e. a unit through which a fluid, such as a fluid preferably a liquid sample can flow e.g. by the use of a pump, capillary forces, gravity or a combination. The flow chip may e.g. be a micro flow chip e.g. where the flow channel in the flow chip has a cross sectional area of about 100.000 μm² or less, such as about 10.000 μm or less, such as about 1000 μm or less.

In one embodiment, the secondary substrate in the form of a circuit board comprises an opening for mounting a flow chip for bringing and optionally withdrawing a fluid preferably a liquid sample from the sensor.

In one embodiment the secondary substrate in form of a printed circuit board also comprises integrated flow channels for bringing and withdrawing a fluid sample to the sensor. The integrated flow channels might also included mixing and filtering areas for mixing and filtering more fluids preferably in the form of liquids.

Thus it should be observed that the inventors have found that in principle all types of flow chips in micro size may be produced using printed circuit boards. By using this production technology a very simple and inexpensive production method has been provided.

The secondary substrate may in principle be of any type of materials e.g. the materials mentioned above for the primary substrate. In one embodiment the secondary substrate is of a material selected from the group epoxy glass; LCP (Liquid Crystal Polymer); polyimide; polycarbonate; polyvinylchloride; ABS (Acrylonitrile-Butadiene-Styrene); ceramic material such as alumina, mullite, glass, silicon, and combinations thereof.

In one embodiment, the secondary substrate is of an epoxy material, such as FR-4 or FR-5 epoxy glass. FR-4 or FR-5 epoxy glass is a group of well known materials and is e.g. marketed by Shell and National Electrical Manufactures Association of USA. FR-4 epoxy resins, which are polyfunctional epoxy resins and in one particular embodiment of the invention is a difunctional brominated epoxy resins. See e.g. Electronic Materials Handbook. ASM International (1989) at pages 534-537.

In one embodiment, the sensor has only one cantilever protruding from the primary substrate, the connecting surface of the primary surface totally surrounds the primary cavity, and the secondary substrate comprises an opening through the substrate to provide access to the cantilever. Thereby a liquid sample can be added to the interaction chamber through the opening in the secondary substrate e.g. in the form of a drop. The sensor in this embodiment may also be part of a dip stick.

In one embodiment, sensor has two or more cantilevers and each cantilever has its own primary cavity. The connecting surface of the primary surface totally surrounds the cavities of the primary surface, and the secondary substrate comprises openings through the substrate to provide access to the cantilevers.

In one embodiment, the sensor has only one cantilever protruding from the primary substrate and into each interaction chamber constituted by a primary cavity and optionally by an opening in the secondary substrate above the primary cavity. The sensor comprises two or more primary cavities, and the secondary substrate comprises openings above said primary cavities. The primary cavities each have a primary connecting surface that totally surrounds it. The liquid sample can be added to the interaction chambers through the openings in the secondary substrate e.g. in the form of drops.

In one embodiment, the primary cavity is in the form of a primary channel section. The primary channel section may preferably extend perpendicular to the protruding direction of the cantilever(s). Thereby an optimal contact between a fluid sample flowing e.g. with laminar flow through the channel section and the cantilever(s) can be obtained.

In this embodiment, the primary connecting surface may e.g. be constituted by the surface along the lengthwise borders of the primary channel section. Thereby the channel section may have an inlet and outlet in opposite ends of the channel sections where the inlet and outlet are in prolongation of the channel sections, and a fluid introduced through the channel section need not be subjected to turns.

In a variation of this embodiment, the primary connecting surface is constituted by the surface along all of the borders of the primary channel section. Thereby the inlet for the fluid may be in the side of the channel section, e.g. through an opening in the secondary substrate.

The secondary substrate may comprise a secondary channel corresponding to the primary channel so that the primary and the secondary channels together form a flow channel section.

In one embodiment, this flow channel section is closed except from an inlet in one of its ends, and an outlet in the other one of its ends. Thereby the flow channel section may have an inlet and optionally an outlet in opposite ends of the flow channel sections where the inlet and outlet are in prolongation of the flow channel sections.

In one embodiment, the flow channel section comprises one or more openings through either the primary or the secondary substrate.

In one embodiment, the primary channel section is in the form of an oblong cavity, the secondary substrate comprises an oblong opening corresponding to the primary channel section, the primary connecting surface surrounding the primary channel section and the secondary connection surface along the oblong opening being mounted to each other to form a flow channel section. In this embodiment, the fluid sample may be introduced through the secondary opening e.g. introduced in one end of the flow channel section and withdrawn from the other end of the flow channel section. This may e.g. be performed by mounting a flow chip above the opening in the secondary substrate.

In one embodiment, the sensor comprises two or more cantilevers protruding from the primary substrate in a row along the length of the primary channel section. In one embodiment, the sensor units comprise two rows of sensor units placed opposite each other and protruding into the channel section.

In one embodiment, the cross sectional area of the channel is about 0.1 mm² or less, such as about 0.05 mm² or less, such as about 0.01 mm² or less. Thereby a fluid flow preferably in the form of a liquid flow through the flow channel will be laminar, and furthermore the amount of fluid necessary for performing a measurement is very low.

Since the dimensions of such sensors of the invention are very small it is actually surprising that this mounting technique has shown to be useful for the production. Thus it has been found to be possible to provide a liquid sensor with a small interaction chamber which is reliable and with very low risk of short circuiting due to liquid interfering with the electrical connections.

Thus in one embodiment, the primary and the secondary connection surfaces are sealed in a liquid tight sealing. This sealing may in principle be provided in any way, but since the dimensions are often small care should be taken not to clog the primary cavity with bonding material and the like.

In one embodiment, the liquid tight sealing comprises metal e.g. a metal sealing ring, polymer, glue or mixtures thereof. When using a metal sealing or sections of a metal sealing ring care should be taken that the metal sealing ring does not come into electrical contact with the primary and secondary connection pads, as this may result in a short circuiting of the system.

In one embodiment, the liquid tight sealing is totally or partly provided by soldering.

In one embodiment, the liquid tight sealing is totally or partly provided by glueing.

In one embodiment, the liquid tight sealing is totally or partly provided by underfiller, such as by underfilling of a polymer e.g. silicone and epoxy resin. Underfiller materials are well known in the art. Useful underfillers are as follows:

Ablebond E1172, Ablebond 7737s and Ablebond E1216, marketed by Emerson & Cuming, Canton, Mass.; Loctite 3563, Loctite 3564 and Loctite 3565 marketed by Loctite (Henkel Loctite Corp), Rocky hill, USA; Delo-Katiobond VE 4530 and Delo-Katiobond VE 4529, marketed by Industrieklebstoffe, Landsberg, Germany; and Chipcoat 8422, marketed by Namics Corporation, Japan.

The underfiller may preferably have suitably viscosity during application so that the underfiller is forced into an intermediate small space between the primary and secondary connection surface using capillary forces.

In one embodiment the one or more cantilevers comprise a capture surface of at least one of its major surfaces. The capture surface may be as described in WO 0066266, PCT/DK/0200779, PCT/DK/0300117, PCT/DK/0300042, PCT/DK/0300086, DK PA 2002 00884, DK PA 2002 01221, and DK PA 2002 00068.

In one embodiment of the sensor according to the invention, the capture surface is a surface of a capture coating comprising a capture layer, wherein said capture layer is a layer comprising a detection ligand, said detection ligand being a member of a specific binding pair wherein said detection ligand preferably is selected from the group consisting of RNA oligos, DNA oligos, PNA oligos, proteins, peptides, hormones, blood components, antigen and antibodies.

The capture coating could in principle have any thickness. If the capture coating is very thick, the sensitivity may be reduced due to the stiffness of the sensor unit. A desired thickness could e.g. be from molecular thickness to 2000 nm, such as up to, 2, 5, 10 or 50 molecule layers, or e.g. between 0.5 nm and 1000 nm, such as between 1 and 500 nm, such as between 10 and 200 nm.

In one embodiment, both or a part of both of the two major sides of the cantilever comprise a capture surface. The capture surfaces may be identical or they may differ from each other e.g. with respect to size of area covered, type of capture molecules and/or concentrations thereof. In one embodiment, the capture surface on one major side of a cantilever is essentially identical,—both with respect to size of area covered, type of capture molecules and concentrations—to the capture surface on the other one of the two opposite major surfaces of the cantilever.

In an additional aspect the invention also relates to a method of improving the quality of the measurements obtained using sensor units based on piezoresistive direct read out systems, such as the sensor systems disclosed in any one of the patent applications WO 0066266, PCT/DK/0200779, PCT/DK/0300117, PCT/DK/0300042, PCT/DK/0300086, DK PA 2002 00884, DK PA 2002 01221, and DK PA 2002 00068, which are hereby for this additional aspect incorporated by reference, and as disclosed above.

The invention in the additional aspect includes the additional feature of grounding the sensor system, by applying a grounded electrode on the sensor e.g. the sensor units or unit(s) or applying a grounded electrode in contact with the flowing fluid. This embodiment is in particular useful in the form of a liquid sensor i.e. adapted for detecting a chemical component in a liquid sample.

During operation of the sensor units in conducting fluids it is thus an advantage to control the electrical potential of the fluid. If this is not carefully obtained e.g. by connecting an electrode to the ground potential, the electrical potential of the fluid might change due to external influences, e.g. static electricity and electrical fields from a computer.

A change in the electrical field of the fluid may influence the number of electrical carriers in the piezo-resistor due to the difference in potential between the fluid and the piezo-resistor in the sensor units. The uncontrolled change in electrical carriers will change the resistance of the piezo-resistor and will be observed as noise and/or drift in the output signal. Using the invention according to the additional aspect this noise and/or drift can be reduced or even avoided.

The potential control can be obtained by integrating an electrode on the sensor e.g. on the respective sensor units or alternatively in the fluid flow system. In order to minimize the variation of electrical carriers due to the influence of the fluid electrical potential, this potential should preferably not differ more than 10 V from the electrical potential of the piezo-resistor of the sensor units.

The electrical potential of the fluid can either be kept constant or it can alternate. In one embodiment the fluid potential alternates with the same frequency as the supply voltage to the piezo-resistors in the sensor units (AC mode). In another embodiment the fluid potential alternates with a different frequency than the supply voltage to the piezo-resistors in the sensor units. In another embodiment the fluid potential is kept constant even though the supply voltage to the piezo-resistors in the sensor units is alternating.

In a third aspect the invention relates to a sensor-reader system including a reader unit and a sensor comprising two or more sensor units, such as 10 or more, such as 50 or more sensor units comprising different capture surfaces. The sensor comprises a bar code which bar code comprises the information about which sensor units a customer has bought access to. The reader unit comprising a reader for reading the bar code and based on this reading the necessary electrical connection to the sensor units that have been paid for are established.

The sensor may e.g. be as the sensors disclosed in any one of the patent applications WO 0066266, PCT/DK/0200779, PCT/DK/0300117, PCT/DK/0300042, PCT/DK/0300086, DK PA 2002 00884, DK PA 2002 01221, and DK PA 2002 00068, which are hereby for this additional aspect incorporated by reference, and as disclosed above.

The respective sensor units are directed as sensor units for s specific target composition e.g. a molecule, and thus the respective sensor units one by one or in groups represents and can be used for performing respective tests.

The reader unit may include a resistance meter for measuring the resistivity over the sensor units that has been paid for, or alternatively the resistance meter may be a separate unit which can be connected to the reader.

By a sensor-reader system according to the third aspect of the invention it is possibly to sell the same type of sensor to different customers with different or overlapping needs. The respective customers thus only get access to the sensor units that they desire and that they wish to buy.

By a sensor-reader system according to the third aspect of the invention it is possibly to mass produce only a few sensor or even only one to satisfy a large number of customers with different needs. Since a mass production is much more economical beneficial than a customised production, the sensor-reader system thus provide a highly beneficial improvement.

The invention in this third aspect also includes a method of providing a customer with desired sensor tests, by using this sensor-reader system as described above. This method including the steps of
i) providing and selling a reader of a sensor-reader system to a customer;
ii) Selling a sensor of the sensor-reader system to the customer, wherein the customer has selected one or more of the tests which is provide for by the sensor units included on the sensor;
iii) providing a sensor of the sensor-reader system and marking it with a bar code corresponding to the tests that have been selected by the customer.

When the customer has bought one reader, he do of course not need to by an extra when buying additional sensors for the sensor-reader system. Thus if the customer has a reader step i) may be skipped.

### BRIEF DESCRIPTION OF DRAWINGS

FIG. 1 is a cross-sectional side view of a sensor according to the invention.

FIG. 2 is a cross-sectional top view of the sensor shown in FIG. 1, where the secondary substrate has been removed.

FIG. 3 is a schematic and exploded perspective view of a sensor according to the invention.

FIG. 4 is a schematic and exploded perspective view of a variation of the sensor shown in FIG. 3.

FIG. 5 is a schematic and exploded perspective view of a variation of the sensor shown in FIGS. 3 and 4.

FIG. 6 is a cross-sectional side view of a variation of the sensor shown in FIG. 1.

FIG. 7 is a top view of a not mounted secondary substrate.

### DETAILED DESCRIPTION OF DRAWINGS

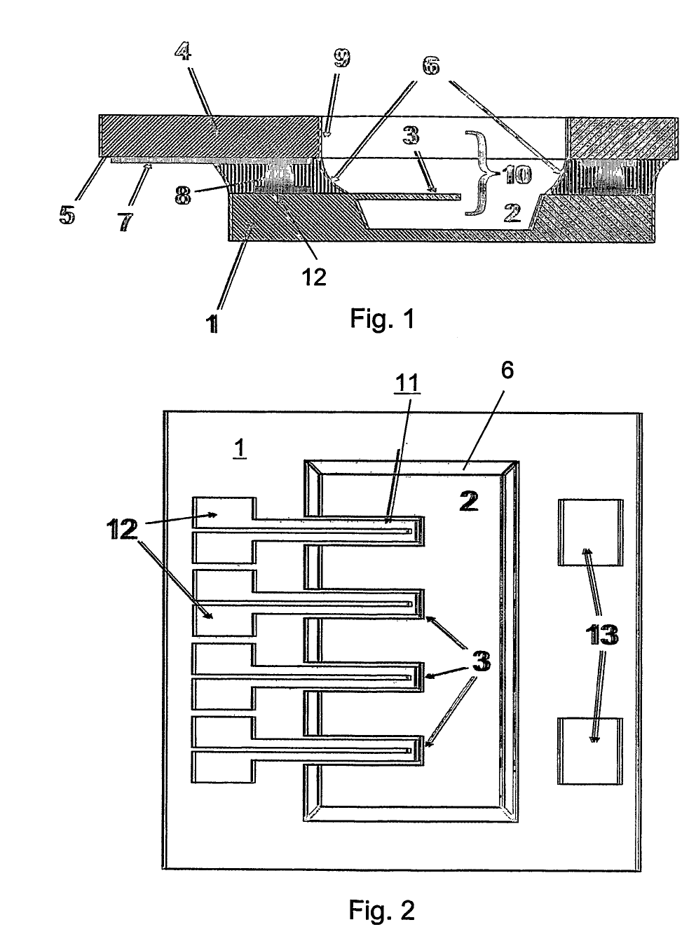

FIG. 1 is a cross-sectional view of a chemical sensor of the invention. The sensor comprises a primary substrate 1 having one or more cantilevers 3 protruding from the primary substrate 1. The cantilevers 3 are suspended above a primary cavity 2 constituted by an etched recess 2.

Furthermore, the primary substrate 1 is provided with bumps 8 for flip-chip assembly. The sensor also comprises a secondary substrate 4 which is provided with electrically conducting pads 7 and an opening 9. In the flip-chip assembly process, the primary substrate 1 is mounted onto the surface 5 of the secondary substrate 4 so that the opening 9 is positioned over the etched recess 2 in the primary substrate. The spacing between the primary substrate 1 and the secondary substrate 4 is filled with a glue 6, preferably an underfiller. The primary connecting surface is constituted by the part of the primary substrate and the part of the primary pads 12 that are in contact with the glue 6 and the bumps 8 that connect the substrates to each other, and the secondary connecting surface is constituted by the secondary pads and the part of the surface 5 of the secondary substrate 4 that are in contact with the glue 6 and the bumps 8 that connect the substrates to each other. After the underfilling process, the opening 9 and the recess 2 form a channel section 10 that can guide a liquid sample to the cantilevers 3. The underfiller 6 separates the electrically conductive pads 7 from the liquid in channel section 10, and therefore the sensor can be used for measuring in conductive liquids without risk of short circuiting.

FIG. 2 shows a cross-sectional top view of the sensor shown in FIG. 1 where the secondary substrate has been removed. The primary substrate 1 in this example, is linked to four cantilevers 3. Each cantilever 3 comprises a piezoresistive strain-gauge 11. The electrical connections to the strain-gauge resistor are provided by the bond pads 12. The primary substrate 1 also comprises extra bond pads 13 for stabilising the semiconductor body during flip-chip mounting.

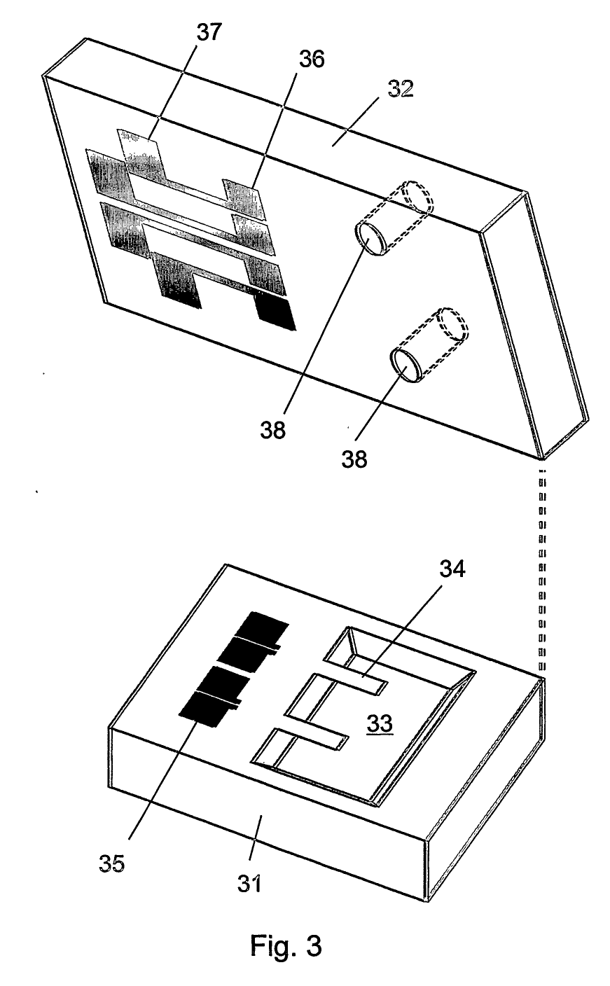

FIG. 3 is a schematic and exploded perspective view of a sensor according to the invention, where the filler material and the bumps that connect the primary substrate 31 and the secondary substrate 32 to each other, are not shown. The primary substrate 31 comprises a cavity 33 in the form of a channel section, and two cantilevers 34 protruding into the cavity 33. The cantilever 34 comprises not shown piezoresistive elements, which are in electrical connection with the primary pads 35. The secondary substrate 32 comprises secondary pads 36 corresponding to the primary pads 35. The secondary pads 36 are each connected to a communication pad 37 through which a not shown power supply can be connected. The secondary substrate 32 further comprises two openings 38 through the secondary substrate 32 positioned above the respective endings of the channel section 33. When mounted together, a sealing is applied between the primary and the secondary connection surface which in this example extends around the border of the primary cavity 33. A liquid sample can thereafter be introduced and withdrawn through the openings 38. The secondary substrate further comprises a not shown cavity to be placed above the cantilevers so that the cantilevers freely can deflect if stress is generated on their surfaces.

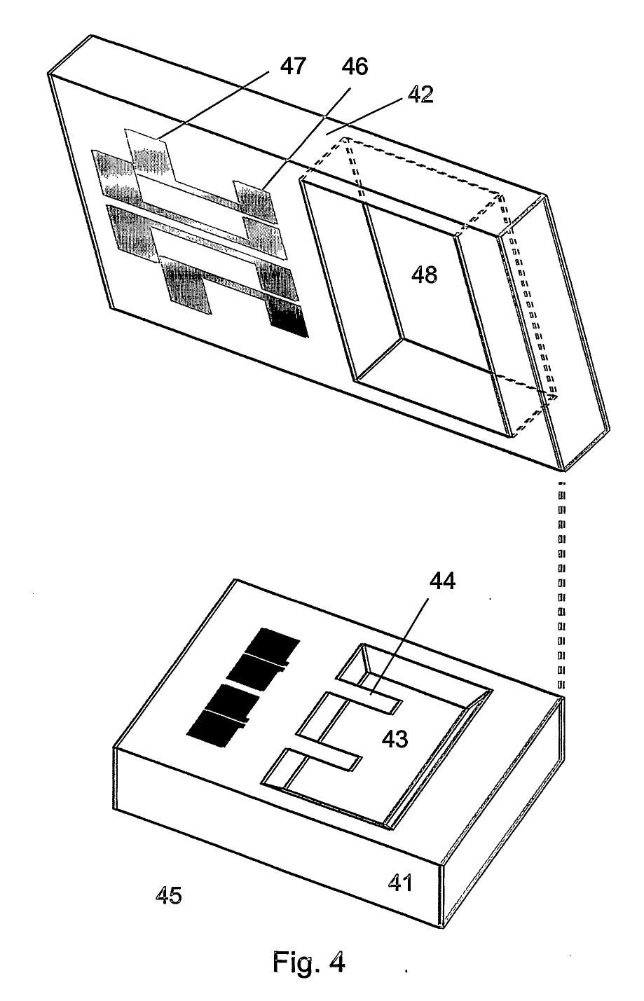

FIG. 4 is a schematic and exploded perspective view of a variation of the sensor shown in FIG. 3, where the filler material and the bumps that connect the primary substrate 341 and the secondary substrate 42 to each other, are not shown. The primary substrate 41 comprises a cavity 43 in the form of a channel section and two cantilevers 44 protruding into the cavity 43. The cantilever 44 comprises not shown piezoresistive elements which are in electrical connection with the primary pads 45. The secondary substrate 42 comprises secondary pads 46 corresponding to the primary pads 45. The secondary pads 46 are each connected to a communication pad 47 through which a not shown power supply can be connected. The secondary substrate 42 further comprises one opening 48 through the secondary substrate 42 positioned above the channel section 43. When mounted together, a sealing is applied between the primary and the secondary connection surface which in this example extends around the border of the primary cavity 43. A liquid sample can thereafter be introduced through the opening 48. The sensor may e.g. be mounted with a flow chip which can provide a partial cover to the middle part of the channel section 43 so that liquid can be introduced and withdrawn via the flow chip and pass along the length of the channel section.

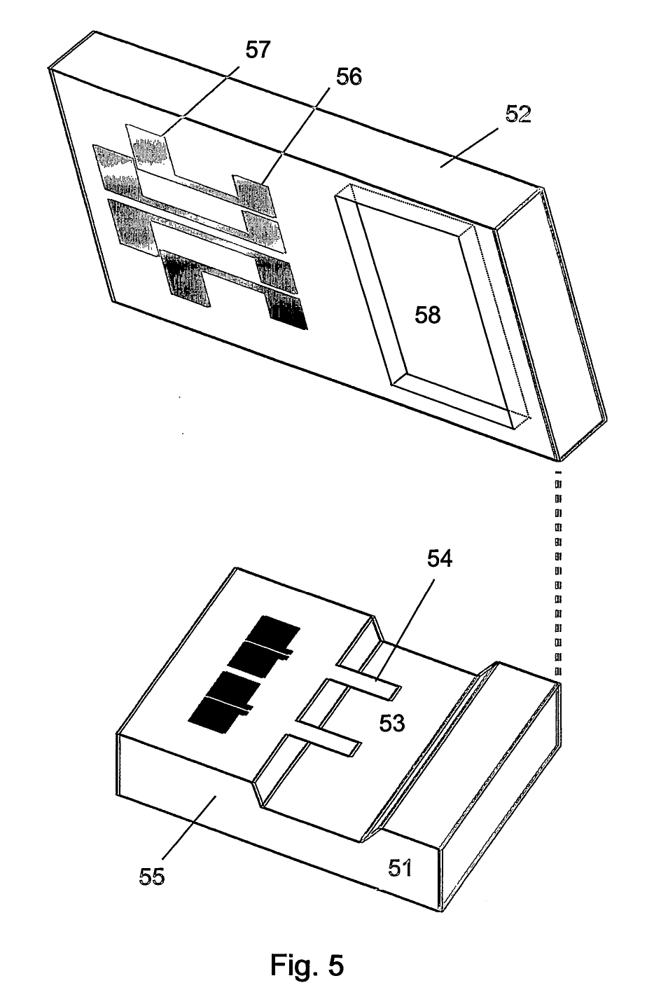

FIG. 5 is a schematic and exploded perspective view of a variation of the sensor shown in FIGS. 3 and 4, where the filler material and the bumps that connect the primary substrate 51 and the secondary substrate 52 to each other, are not shown. The primary substrate 51 comprises a cavity 53 in the form of a channel section passing across the primary substrate 51, and two cantilevers 54 protruding into the cavity 53. The cantilever 54 comprises not shown piezoresistive elements which are in electrical connection with the primary pads 55. The secondary substrate 52 comprises secondary pads 56 corresponding to the primary pads 55. The secondary pads 56 are each connected to a communication pad 57 through which a not shown power supply can be connected. The secondary substrate further comprises a cavity 58 arranged to be placed above the cantilevers so that the cantilevers freely can deflect if stress is generated on their surfaces.

When mounted together, a sealing is applied between the primary and the secondary connection surface which in this example extends along the borders of the primary cavity 53 following the length direction of the channel section 53. Two openings for introducing and withdrawing of liquid sample will thereby be formed in prolongation of the channel endings.

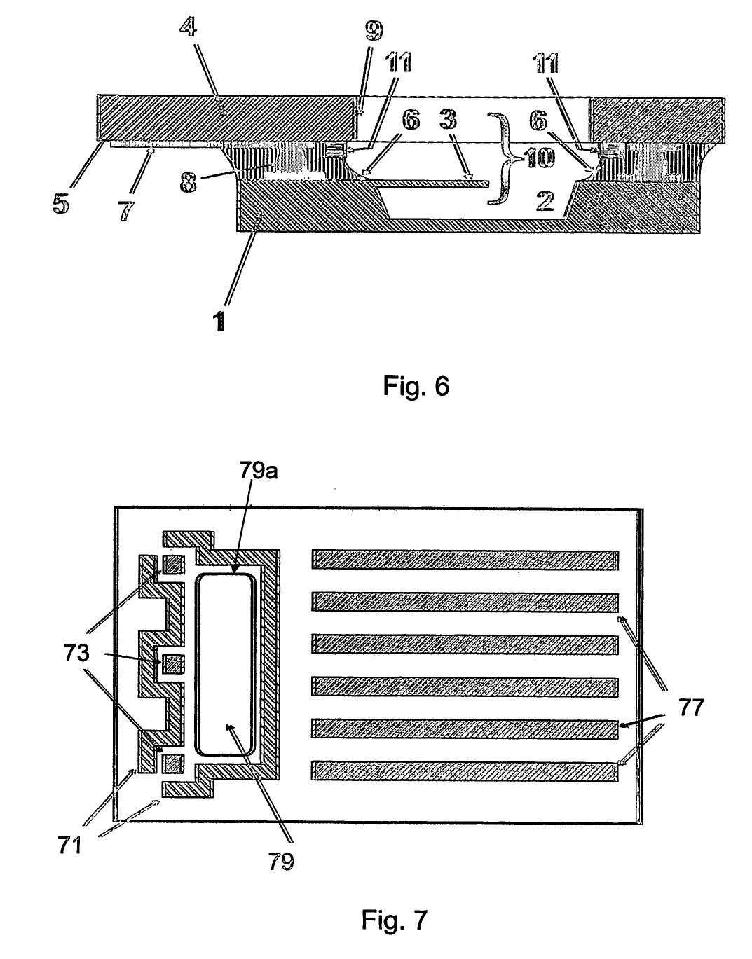

FIG. 6 is a cross-sectional side view of a variation of the sensor shown in FIG. 1, wherein the sensor further comprises a barrier wall 11. The remaining parts of the sensor are as described above for FIG. 1.

The barrier wall 11 prevents the underfiller from flowing into the primary cavity 2.

FIG. 7 is a top view of a not mounted secondary substrate seen from the face adapted to be mounted together with a primary substrate. The secondary substrate comprises an opening 79, electrically conducting pads 77, pads for a stabilising connection 73 and a barrier wall 71 in two sections. As seen the distance between the border line 79a of the opening 79 and the barrier wall may vary. The border line 79a preferably corresponds to the borderline of the primary cavity of the primary substrate to which the secondary substrate is supposed to be mounted with.

1-26. (canceled)

27. A chemical sensor comprising at least one sensor unit, a primary and a secondary substrate, the primary substrate comprises a primary cavity and a primary connecting surface at least partly surrounding said cavity, the at least one sensor unit is in the form of cantilevers, each comprising a piezoresistive element, said at least one sensor unit is protruding from the primary substrate and into the cavity of said primary substrate, the piezoresistive element or elements being electrically connected to primary connecting pads on the primary connecting surface, the secondary substrate comprises secondary connecting pads corresponding to the primary connecting pads, on a secondary connecting surface corresponding to the primary connecting surface, said primary connecting surface and said secondary connecting surface being mounted to each other so that said primary connecting pads and said secondary connecting pads are directly mounted to each other.

28. A chemical sensor according to claim 27 wherein the sensor has one cantilever protruding from the primary substrate, the primary connecting surface of the primary surface totally surrounds the primary cavity, and the secondary substrate comprises an opening through the substrate to provide access to the cantilever.

29. A chemical sensor according to claim 28 wherein the sensor has at least two cantilevers, each cantilever has its own primary cavity, the primary connecting surface of the primary surface totally surrounds the cavities of the primary surface, and the secondary substrate comprises openings through the substrate to provide access to the cantilevers.

30. A chemical sensor according to claim 27 wherein the primary cavity is in the form of a primary channel section, said primary channel section extending perpendicular to the protruding direction of at least one cantilever.

31. A chemical sensor according to claim 30 wherein the primary connecting surface is constituted by the surface along the lengthwise borders of the primary channel section.

32. A chemical sensor according to claim 30 wherein the primary connecting surface is constituted by the surface along all of the borders of the primary channel section.

33. A chemical sensor according to claim 30 wherein the secondary substrate comprises a secondary channel corresponding to the primary channel so that the primary and the secondary channels together form a flow channel section.

34. A chemical sensor according to claim 33 wherein the flow channel section is closed except from an inlet in one of its ends and an outlet in the other one of its ends.

35. A chemical sensor according to claim 33 wherein the flow channel section comprises at least one openings through either the primary or the secondary substrate.

36. A chemical sensor according to claim 32 wherein the primary channel section is in the form of an oblong cavity, the secondary substrate comprises an oblong opening corresponding to the primary channel section, the primary connecting surface surrounding the primary channel section and the secondary connection surface along the oblong opening being mounted to each other to form a flow channel section.

37. A chemical sensor according to claim 30 wherein the sensor comprises at least two cantilevers protruding from the primary substrate along the length of the primary channel section.

38. A chemical sensor according to claim 27 wherein the primary connecting surface comprises a barrier line extending partly or totally around the primary cavity, said barrier line being in the form of a barrier line selected from the group of a) a barrier wall, b) a barrier ditch and c) both a barrier wall and a barrier ditch.

39. A chemical sensor according to claim 27 wherein the secondary connecting surface comprises a cavity or an opening in the secondary substrate, said secondary substrate further comprising a barrier line extending at least partly around the cavity or at least partly around the opening in the secondary substrate.

40. A chemical sensor according to claim 27 wherein the primary and the secondary connection surfaces are sealed in a liquid tight sealing.

41. A chemical sensor according to claim 40 wherein the liquid tight sealing comprises materials are chosen from metal, polymer, glue and mixtures thereof.

42. A chemical sensor according to claim 40 wherein the liquid tight sealing is at least partly provided by soldering.

43. A chemical sensor according to claim 40 wherein the liquid tight sealing is at least partly provided by underfilling.

44. A chemical sensor according to claim 27 wherein the secondary substrate is of a ceramic material.

45. A chemical sensor according to claim 27 wherein the secondary substrate is a printed circuit board.

46. A chemical sensor according to claim 27 wherein the secondary substrate is a micro chip.

47. A chemical sensor according to claim 27 wherein said primary substrate is based on silicon, said primary cavity being in the form of an etched cavity forming a recess under at least one cantilever.

48. A chemical sensor according to claim 27 wherein the sensor comprises a grounded electrode to ground the potential of a conductive fluid in at least one of the cavities of the primary substrate.
